SEQUENCE LISTING (2 sequences)

SEQ ID NO: 1 - protein, 57 aa, Homo sapiens
IPPHVQKSVNNDMIVTDNNGAVKFPQLCKFCDVRFSTCDNQKSCFSKVHYEGKKKAW

SEQ ID NO: 2 - protein, 286 aa, Artificial Sequence
  fusion protein
IPPHVQKSVNNDMIVTDNNGAVKFPQLCKFCDVRFSTCDNQKSCFSKVHYEGKKKAWRSDKTHTCPPCPAPELLGGPSVFLFPPKPKDQLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

(19) United States
(12) Patent Application Publication
(10) Pub. No.: US 2023/0365654 A1
(43) Pub. Date: Nov. 16, 2023

(54) METHOD OF TREATMENT OF FIBROSIS AND WOUND HEALING

(71) Applicants: Consejo Nacional de Investigaciones Científicas y Técnicas, Ciudad Autónoma de Buenos Aires (AR); RADBIO USA, Inc, Wilmington, DE (US)

(72) Inventors: Ana Romo, Buenos Aires (AR); Ricardo Alfredo DEWEY, La Plata (AR)

(73) Assignees: Consejo Nacional de Investigaciones Científicas y Técnicas, Ciudad Autónoma de Buenos Aires (AR); RADBIO USA, Inc, Wilmington, DE (US)

(21) Appl. No.: 18/030,089

(22) PCT Filed: Oct. 4, 2021
(86) PCT No.: PCT/US2021/053297

§ 371 Date: Apr. 14, 2023

Related U.S. Application Data
(60) Provisional application No. 63/087,375, filed on Oct. 5, 2020.

(51) Int. Cl. C07K 14/71; A61P 1/16; A61K 8/64; A61Q 19/08; A61P 17/02

(52) U.S. Cl. CPC C07K 14/71; A61P 1/16; A61K 8/64; A61Q 19/08; A61P 17/02; C07K 2319/30

(57) ABSTRACT

A method of treating different fibrosis such as liver fibrosis, lung fibrosis or skin fibrosis, wound healing and skin rejuvenation is disclosed by administering a fusion protein having a soluble type II TGF-receptor bound to a stabilizing agent.

### CROSS REFERENCE TO RELATED APPLICATION

This application is a national stage entry of PCT/US21/053297 filed Oct. 4, 2021, under the International Convention claiming priority over US Provisional Application No. 63/087,375 filed Oct. 5, 2020.

### BACKGROUND OF THE INVENTION

Fibrosis is the excessive accumulation of extracellular matrix (ECM) that can cause distortion of tissue architecture and loss of organ function. This pathology commonly results from a wound-healing response produced by chronic or repeated tissue damage or injury, regardless of the underlying etiology, and can occur in virtually any organ or solid tissue. A wide range of prevalent chronic diseases can lead to fibrosis, including diabetes, hypertension, viral and non-viral hepatitis, heart failure and cardiomyopathy, idiopathic lung disease, scleroderma, and cancer. Fibrosis resulting from these and other diseases can lead to liver, lung, kidney, heart, or other vital organ failure, as excessive ECM replaces and disrupts parenchymal tissue. Consequently, severe fibrosis is estimated to account for up to 45% of all deaths in the developed world. Current therapies for fibrosis are few and of limited efficacy. Therefore, there is an urgent need to understand how fibrosis can regress and to identify potential new therapeutic approaches. (Jun J I, Lau L F. J Clin Invest. 2018; 128 (1): 97-107.

The skin is the largest organ system in the body. As such, it plays a critical role in protecting against mechanical forces and infections, fluid imbalance, and thermal dysregulation. At the same time, it allows flexibility to allow joint function in some areas of the body and a more rigid fixation to make it difficult to displace the sole of the palm or the foot. Many cases lead to inadequate wound healing requiring medical intervention. Chronic conditions such as diabetes mellitus or peripheral vascular disease can lead to poor wound healing. Acute trauma, such as skin tearing or large-scale thermal injuries, are followed by a loss of function of the skin organs, which makes the body vulnerable to infection, thermal dysregulation and fluid loss. (Sorg H, et al. Eur Surg Res. 2017;58(1-2):81-94).

Wound healing therapies can be broadly classified into traditional and modern therapies, with varying levels of efficacy, clinical acceptance, and side effects. Traditional therapies have been used for many centuries primarily by rural populations in developing countries. These therapies generally involve the use of compounds derived from herbs and animals, living organisms, silver, and traditional dressings. On the other hand, modern therapies include the use of grafts, modern dressings, bioengineered skin substitutes, and cell dressings/growth factor therapies. However, the regeneration of healthy and functional skin remains a great challenge due to its multilayer structure and the presence of different types of cells within the extracellular matrix in an organized way (Pereira R F, Bártolo P J. Adv Wound Care (New Rochelle). 2016;5(5):208-229).

There are two primary processes of skin aging: intrinsic and extrinsic. Variations in the individual genetic background intrinsically govern aging, which occurs as time passes. By definition, this form of aging is inevitable and, therefore, apparently not subject to manipulation through changes in human behavior. On the contrary, extrinsic aging is generated by factors of external origin that are introduced to the human body, such as smoking, excessive alcohol consumption, poor nutrition and chronic exposure to the sun. Of these external factors, sun exposure is considered to be by far the most significantly damaging to the skin. In fact, 80% of facial aging is believed to be due to chronic exposure to the sun. The aging process, both intrinsic and extrinsic, is also believed to be influenced by the formation of free radicals, also known as reactive oxygen species. Loss of collagen is considered the characteristic histological finding of aging skin. Wrinkles and pigmentary changes are directly associated with photoaging and are considered its most prominent skin manifestations. Such photo damage represents the skin signs of premature aging.

Furthermore, the deleterious consequences of chronic sun exposure, specifically various forms of photoinduced skin cancer, are also related to acute and chronic sun exposure. The only known strategies aimed at preventing photoaging include avoiding the sun, using sunscreens to block or reduce skin exposure to UV radiation, using retinoids to inhibit collagenase synthesis and promote collagen production, and using antioxidants, particularly in combination, to reduce and neutralize free radicals. (Bauman L. J Pathol 2007; 211: 241-251).

There are different attempts for the prevention or treatment of fibrosis, for example: patent EP 2 566 967 B1 discloses a cadherin-11 antagonist for treating fibrosis exhibiting cadherin-11 activity. In patent EP 2 010 551 B1 the use of a GBA2 inhibitor is disclosed where the inhibitor can be N-[5′-(adamantan-1′-yl-methoxy)-pentan]-1-deoxynojirimycin, N-[5′-(adamantan-1′-yl-methoxy)-pentan]-L-ido-1-deoxynojirimycin, N-[5′-neopentyloxy-pentan]-1-deoxynojirimycin and N-[5′-neopentyloxy-pentan]-L-ido-1-deoxynojirimycin, wherein said inhibitor is used to prepare a drug for the treatment of cystic fibrosis. In patent document EP 1 997 507 A2 a composition for the treatment of an annulus fibrosis defect is disclosed comprising an angiogenic factor and a transporter. The composition stimulates angiogenesis and re-vascularization. U.S. Pat. No. 9,937,133 B2 discloses an aldehyde dehydrogenase inhibitor for the treatment of fibrosis. U.S. Pat. No. 10,106,603 B2 discloses the use of an agent that inhibits the action of Interleukin 11.

### SUMMARY OF THE INVENTION

A method is provided for treating fibrosis in a mammal in need thereof, wherein the method comprises administering to said mammal a therapeutically effective amount of a fusion protein comprising a soluble TGF-β type II receptor (TBRII-SE) bound to a stabilizing agent. The soluble receptor can be soluble TGF-β type II of SEQ ID N° 1 or peptides having 85%, 86%, 87%; 88%, 89%, 90%, 91%, 92%, 93%, 94%, 95%, 96%, 97%, 98%, or 99% sequence identity with SEQ ID No. 1. The stabilizing agent it can be any peptide that stabilizes the fusion protein. In a preferred embodiment the stabilizing agent is the Fc domain of an immunoglobulin (Ig). In another more preferred embodiment the stabilizing agent is the Fc domain of human immunoglobulins G (IgG). The fibrosis can be any fibrosis of any origin, in a preferred embodiment the fibrosis is liver fibrosis, lung fibrosis or skin fibrosis. If the mammal is a human being, the fibrosis may be due, but not limited to, the following disorders and/or diseases nonalcoholic steatohepatitis (NASH), Scleroderma/Systemic Sclerosis (SSc), idiopathic pulmonary fibrosis, and Primary biliary cholangitis (PBC).

Also provided is a composition for the treatment of fibrosis comprising a soluble TGF-β type II receptor fusion protein (SEQ ID No. 1) linked to a stabilizing agent; and pharmaceutically acceptable excipients. In a preferred embodiment the stabilizing agent is an immunoglobulin Fc domain, for example the Fc domain of human IgG. In a preferred embodiment the fusion protein comprises an amino acid sequence that is 85%, 86%, 87%; 88%, 89%, 90%, 91%, 92%, 93%, 94%, 95%, 96%, 97%, 98%, or 99% identity with the sequence shown in SEQ ID No. 2.

A method is provided for healing wounds in a mammal in need thereof, wherein the method comprises administering to said mammal a therapeutically effective amount of a fusion protein comprising soluble TGF-β type II receptor bound to a stabilizing agent. The soluble receptor can be soluble TGF-β type II of SEQ ID N° 1 or peptides having 85%, 86%, 870%; 88%, 89%, 90%, 91%, 92%, 93%, 94%, 95%, 96%, 97%, 98%, or 99% sequence identity with SEQ ID No. 1. The stabilizing agent it can be any peptide that stabilizes the fusion protein. In a preferred embodiment the stabilizing agent is the Fc domain of an immunoglobulin (Ig), for example and without limiting it the stabilizing agent is the Fc domain of human IgG.

A wound healing composition is provided comprising a fusion protein having an endogenous soluble TGF-β type II receptor (TβRII-SE) bound to a stabilizing agent; and pharmaceutically acceptable excipients.

A method is provided for decreasing the aging of the skin of a human being, wherein the method comprises administering to said human an effective amount of a fusion protein comprising a soluble TGF-β type II receptor bound to a stabilizing agent. In a preferred embodiment the stabilizing agent is the Fc domain of an immunoglobulin, for example the Fc domain of human IgG. In a preferred embodiment the fusion protein comprises an amino acid sequence that is 85%, 86%, 87%; 88%, 89%, 90%, 91%, 92%, 93%, 94%, 95%, 96%, 97%, 98%, or 99% identity with the sequence shown in SEQ ID No. 2.

A cosmetic composition is provided for decreasing the aging of human skin comprising a fusion protein comprising a soluble TGF-β type II receptor linked to a stabilizing agent; and acceptable excipients.

The compositions of the invention can be applied by any of the known routes, for example intradermal, subcutaneous, intravenous, intramuscular, in situ, dermal, deposited on a surface, for example skin. The compositions of the invention can be formulated as creams, gels, liquids, pills, capsules, injectables, lotions, controlled release compositions, patches or other known forms.

### BRIEF DESCRIPTION OF THE FIGURES

FIG. 1: Daily body weight determinations of mice treated with the TβRII-SE/Fc fusion protein, Nintedanib and vehicle (control). Nintedanib vs Vehicle * P<0.05; ** P<0.01; *** P<0.001.

FIG. 2: Representative photomicrographs of skin tissue stained with H&E show the effect of treatment with the fusion protein TβRII-SE/Fc and Nintedanib on dermal thickness (white arrows) and dermal white adipose tissue (dWAT) (black arrows) in mice with bleomycin-induced skin fibrosis (A). Quantification of dermal thickness (B) and dWAT thickness (C). #P<0.1; ** P<0.01.

FIG. 3: Representative photomicrographs of Masson's trichrome stained skin tissue show the antifibrotic effect of treatment with the TβRII-SE/Fc fusion protein and Nintedanib in mice with bleomycin-induced skin fibrosis (A). Quantification of the fibrotic area (B). #P<0.1; n.s .: not-significant.

FIG. 4: Representative photomicrographs of Masson's trichrome stained lung tissue show bleomycin-induced lung fibrosis in animals treated with vehicle, TβRII-SE/Fc fusion protein, and Nintedanib (A). The quantification of pulmonary fibrosis assessed as the Ashcroft index indicates that treatments with the TβRII-SE/Fc fusion protein and Nintedanib tend to reduce fibrosis compared to the vehicle group (control) (B). #P<0.1.

FIG. 5: Transaminase levels in rats with CCl4-induced liver fibrosis, after two weeks of treatment with the TβRII-SE/Fc fusion protein; ALT (A) and AST (B). * P<0.05; ** P<0.01.

FIG. 6: Histological effects of four weeks of treatment with the TβRII-SE/Fc fusion protein in a model of CCl4-induced liver fibrosis in rats. Treatment with TβRII-SE/Fc reduced the fibrotic index (A) and the activity index of non-alcoholic fatty liver disease (NAFLD) (B), decreased liver inflammation (C), and the number of oval cells (D), indicating a positive effect on the improvement of liver structure. #P<0.1; * P<0.05; ** P<0.01.

FIG. 7: The effects of the TβRII-SE/Fc fusion protein on wound repair in human skin explants are shown. Representative sections stained with Masson's trichrome solution after 4 days of treatment. Arrows indicate the length of the neoepidermis (A). Effects of TβRII-SE/Fc after 4 days of treatment on the growth of the neoepidermis (B). Quantification of explants that showed complete closure of the wound on day 8 of treatment (C). Effects of the TβRII-SE/Fc fusion protein on the differentiation of the neoepidermis in injured skin explants (D). * P<0.05.

FIG. 8: The anti-aging effect of the TβRII-SE/Fc fusion protein is shown by increasing the production of type I collagen in human skin explants. Representative photomicrographs of the increase in collagen I expression evidenced by immunofluorescence after 4 days of treatment with the TβRII-SE/Fc fusion protein (A). Quantification of fluorescence due to the presence of collagen in explants treated for 4 and 8 days with Vehicle (control) or TβRII-SE/Fc (B). *** P<0.001.

FIG. 9: Representative diagram of lung sampling in the pulmonary fibrosis model.

FIG. 10: Representative diagram of the realization of mechanical lesions on rectangular human skin explants using a 3.5 mm diameter biopsy punch.

### DETAILED DESCRIPTION OF THE INVENTION

The term “TβRII-SE/Fc” and “TβRII-SE/Fc fusion protein” have the same meaning and are interchangeable. In a preferred but limited embodiment the fusion protein has an amino acid sequence that is 85%, 86%, 87%; 88%, 89%, 90%, 91%, 92%, 93%, 94%, 95%, 96%, 97%, 98%, or 99% identity with the sequence shown in SEQ ID N° 1

In one embodiment the Fc domain of human immunoglobulins is the Fc domain of human IgG. In another preferred embodiment the Fc domain of human IgG may have a point mutation to eliminate an N-glycosylation site, thereby obtaining a deglycosylated Fc domain, for example the Asn297Gly change in the IgG1 heavy chain.

A wound is understood, without limiting it, to an injury that may or may not be bleeding, which occurs in the outer tissues of the body as a result of a cut, a shot, a pressure, a rub, an operation, diabetes, etc.

Healing is understood as the biological process of tissue repair.

Aging of the skin is understood to be two processes: the intrinsic process that occurs in it as time passes or extrinsic produced by factors of external origin that are introduced into the human body, such as smoking, excessive alcohol consumption, poor diet, and chronic exposure to the sun. The aging process, both intrinsic and extrinsic, is influenced by the formation of free radicals, also known as reactive oxygen species. Loss of collagen is considered the characteristic histological finding of aging skin. Wrinkles and pigmentary changes are directly associated with photoaging and are considered its most prominent skin manifestations. Such photodamage represents the skin signs of premature aging. In addition, the deleterious consequences of chronic sun exposure, specifically various forms of photoinduced skin cancer, are also related to acute and chronic sun exposure. It is understood by slowing down skin aging to reverse these skin manifestations.

The stabilizing agent can be any stabilizing agent that increases the half-life of the fusion protein in vivo, by increasing receptor-mediated recycling of the protein, preventing renal clearance, and decreasing enzymatic degradation.

The fusion protein can comprise a ligand between both parts.

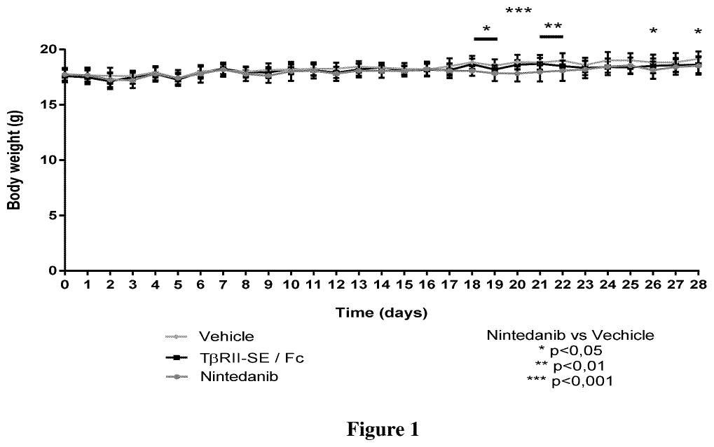

In the case of cutaneous fibrosis, the mean body weight of the Nintedanib group was significantly lower than the weight of the vehicle group (control) from day 18 to 22 and on day 26 and day 28 (FIG. 1). There were no significant differences in mean body weight on any day during the study period between the vehicle group and the group treated with the TβRII-SE/Fc fusion protein. There were no dead animals in all groups during the rearing period. None of the animals showed deterioration in their general condition.

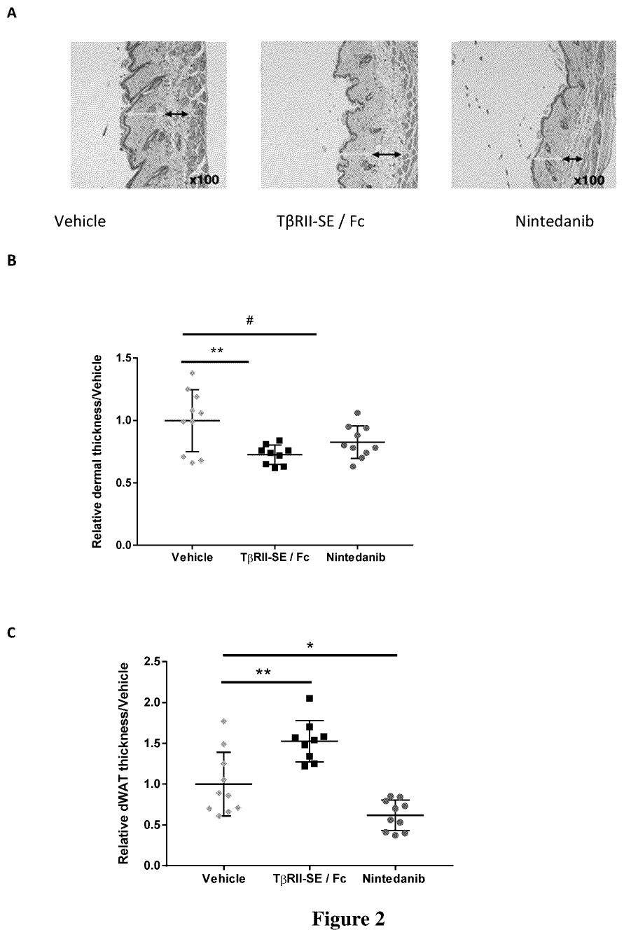

Representative photomicrographs of quantified H&E stained skin sections (FIG. 2) showed a statistically significant decrease in dermal thickness along with a notable expansion of the dermal white adipose tissue (dWAT) layer in the group treated with the protein of TβRII-SE/Fc fusion compared to the vehicle group.

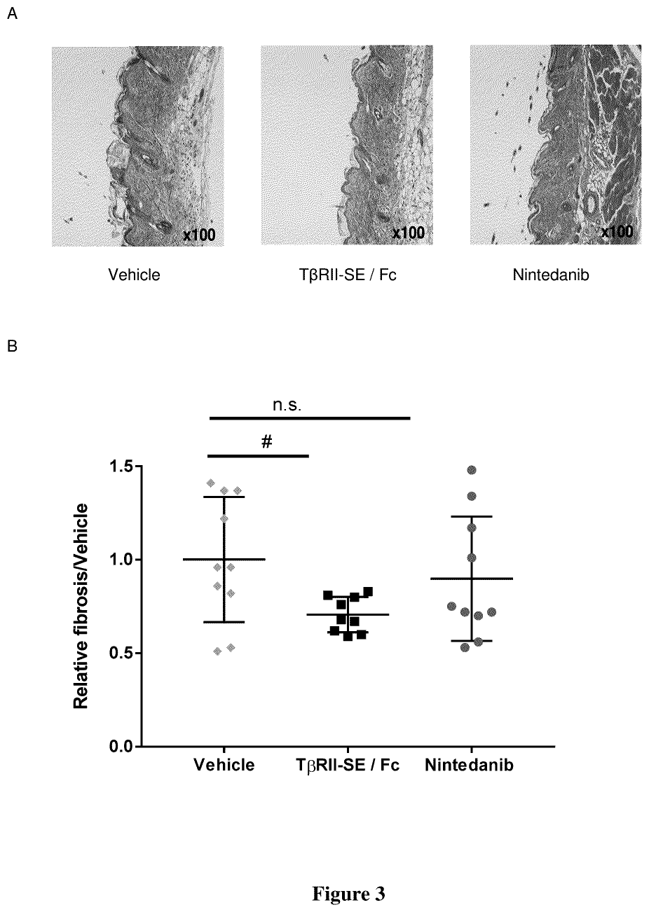

Representative photomicrographs of Masson's trichromic stained skin and quantification (FIG. 3) showed that the area of fibrosis in the group treated with the TβRII-SE/Fc fusion protein tended to decrease compared to the vehicle group. There were no significant differences in the area of fibrosis between the vehicle group and the Nintedanib treated group.

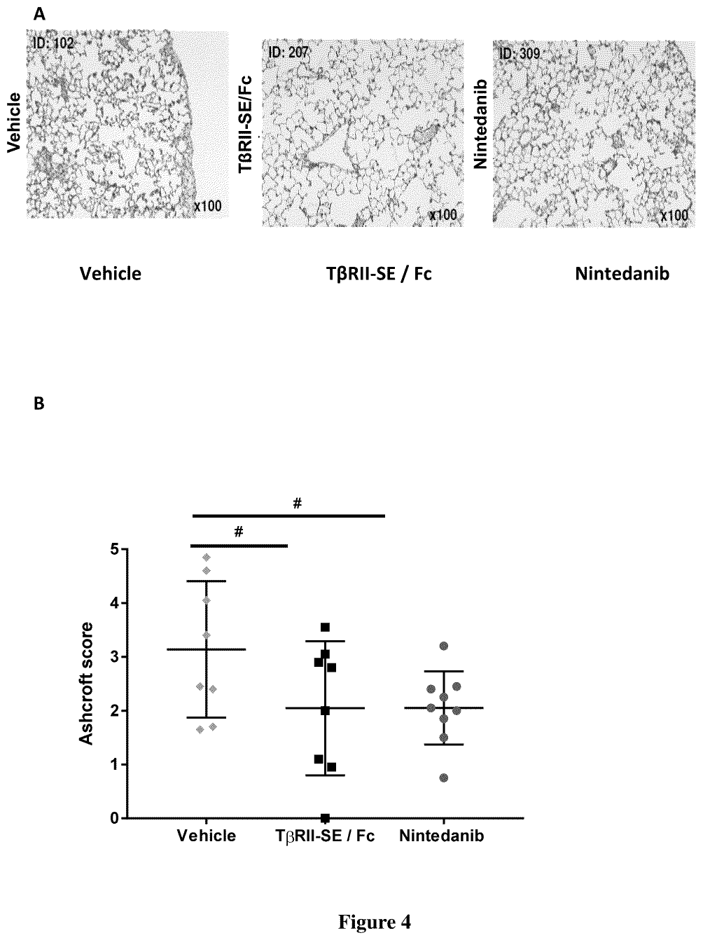

In cases of pulmonary fibrosis, photomicrographs representing Masson's trichrome stained lung sections showed a decrease in pulmonary fibrosis in animals treated with the TβRII-SE/Fc fusion protein (FIG. 4). The Ashcroft score in the TβRII-SE/Fc and Nintedanib groups tended to decrease compared to the vehicle group (FIG. 4).

As evidenced by H&E staining and Masson's trichrome stain, epidermal hypertrophy and skin fibrosis were established in the vehicle-treated group.

Treatment with TβRII-SE/Fc showed a significant decrease in dermal thickness and a tendency to decrease in the area of fibrosis compared to the vehicle treated group (control group). Furthermore, TβRII-SE/Fc treatment showed a significant increase in the dWAT layer.

In conclusion, these results suggested that the TβRII-SE/Fc fusion protein has the potential to suppress skin fibrosis and regenerate the dWAT layer.

In the vehicle group (control group) the establishment of pulmonary fibrosis was observed, as demonstrated by the Masson trichrome staining tests and the Ashcroft score.

Treatment with TβRII-SE/Fc and Nintedanib showed a decreasing trend in the Ashcroft score compared to the vehicle group.

In conclusion, treatment with TβRII-SE/Fc showed a decreasing trend in Ashcroft score compared to the vehicle group, suggesting that TβRII-SE/Fc has antifibrotic effects in pulmonary fibrosis.

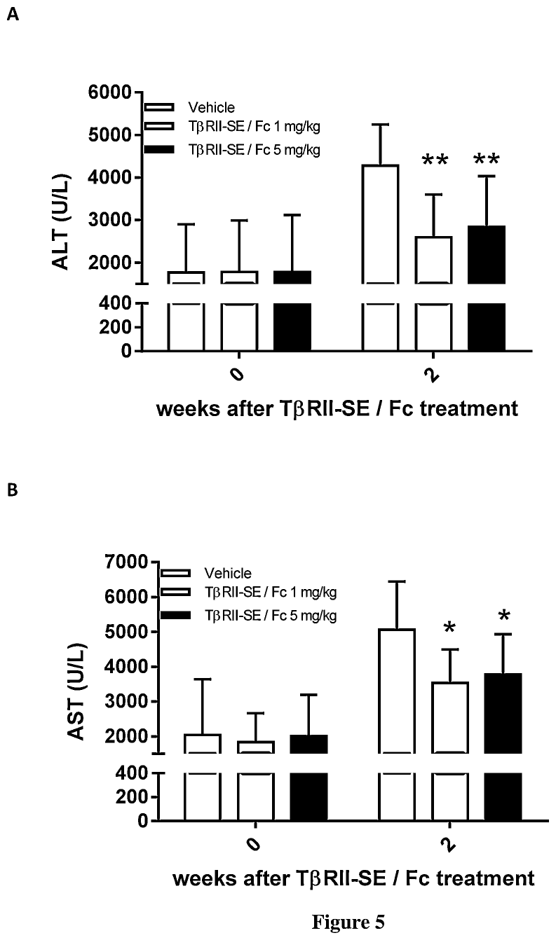

In the tests carried out in the liver fibrosis model, it was shown that in the treatment with CCl4, the levels of ALT and AST in the serum of the rats increased dramatically (FIG. 5). After only 2 weeks of treatment with the TβRII-SE/Fc fusion protein (4 weeks after CCl4 treatment), animals that received 5 mg/kg or 1 mg/kg showed statistically significant differences in ALT/AST compared to the model group or control group (FIG. 5) analyzed by bidirectional ANOVA. Samples were taken 24 h after the last dose of CCl4.

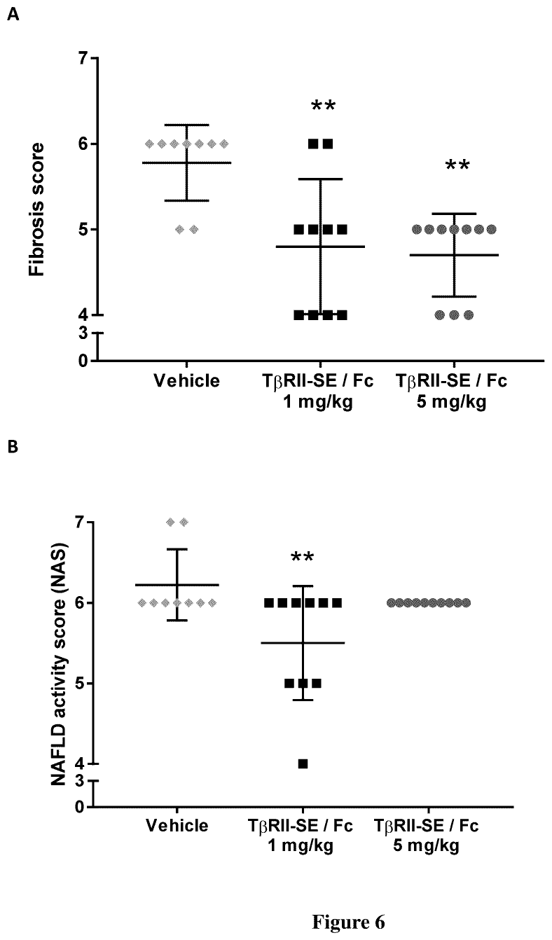

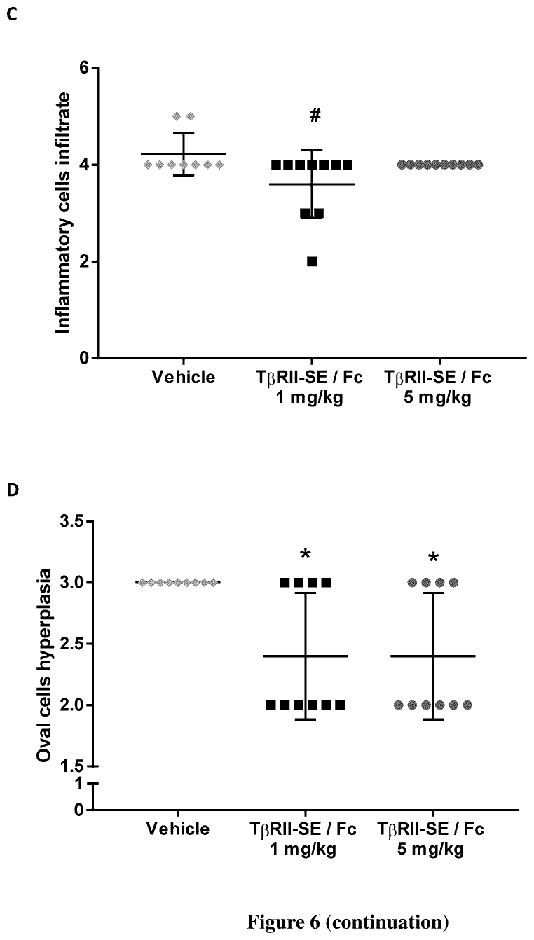

Statistically significant differences in fibrosis score, non-fatty liver disease index (NAFLD), inflammatory cell infiltrate, and oval cell hyperplasia (#P<0.1, * P<0.05, ** P<0.01) (FIG. 6).

Analysis of H&E stained liver sections allowed the evaluation of various histological parameters. Kruskal-Wallis analysis followed by Dunn's test indicated statistically significant differences in fibrosis score, inflammatory cell infiltrate, and oval cell hyperplasia (* P<0.05, ** P<0.01) (FIG. 6).

Treatment with CCl4 dramatically raised the ALT/AST level and promoted liver fibrosis. Treatment with TβRII-SE/Fc significantly reduced ALT/AST levels after 2 weeks of treatment of 2 doses per week (4 doses).

Treatment with TβRII-SE/Fc significantly reduced fibrotic score, inflammatory cell infiltrate, and oval cell hyperplasia, indicating improvement in liver fibrosis and partial restoration of liver architecture.

The results of tests on the wound repair and anti-aging model in live human skin explants were analyzed. The results showed that TβRII-SE/Fc was well tolerated by human skin explants after 4 and 8 days of treatment at 5 and 200 μg/mL.

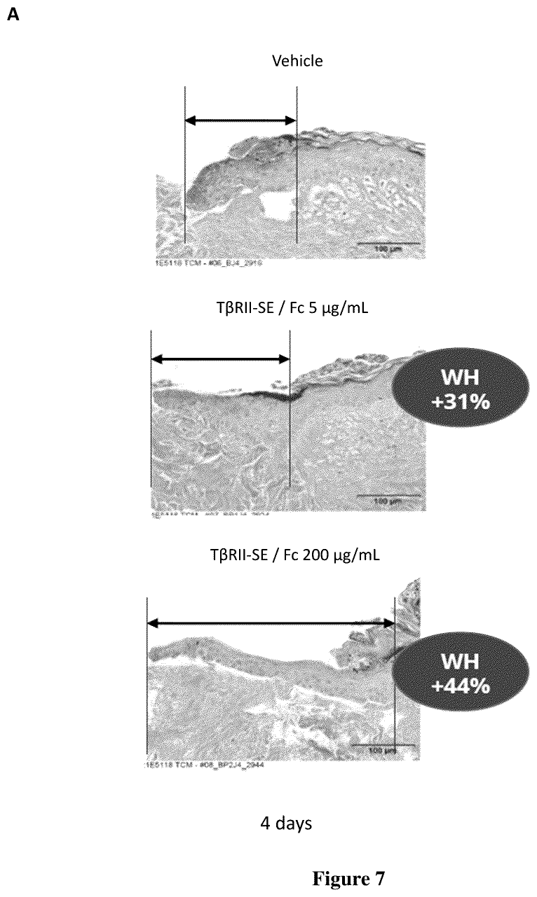

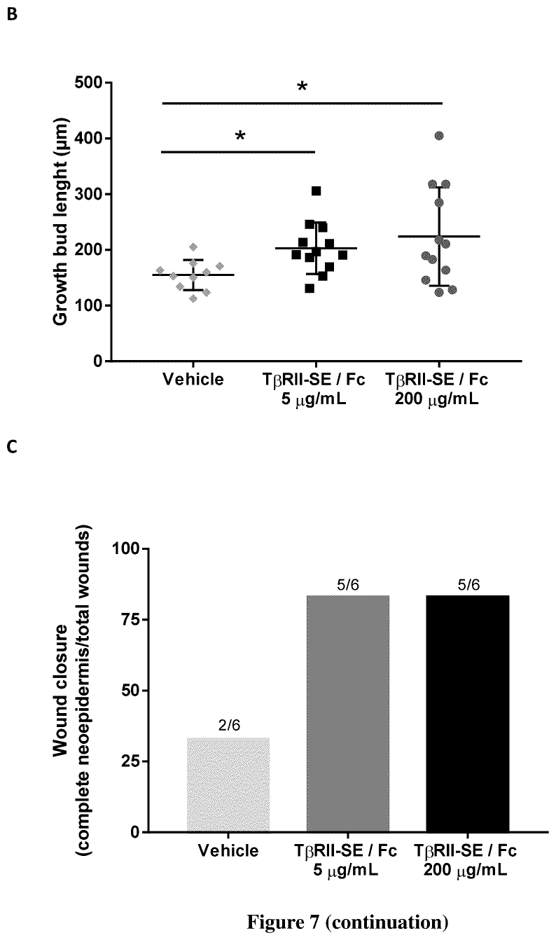

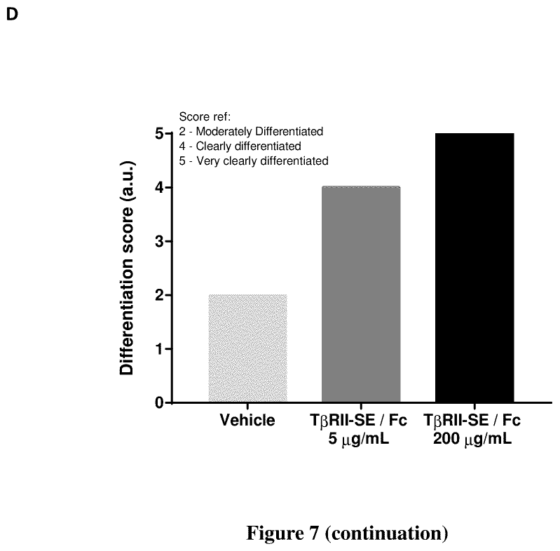

On the other hand, on day 4, the TβRII-SE/Fc fusion protein significantly increased wound healing by 31% (5 μg/ml) and 44% (200 μg/ml) (FIG. 7A). On day 8, 5 of the 6 explants treated with TβRII-SE/Fc (either 5 or 200 μg/ml) showed complete wound healing, while only 2 of the 6 control explants closed the wound (FIG. 7). Furthermore, TβRII-SE/Fc showed an outstanding effect on the differentiation of the neoepidermis on day 8 (FIG. 7).

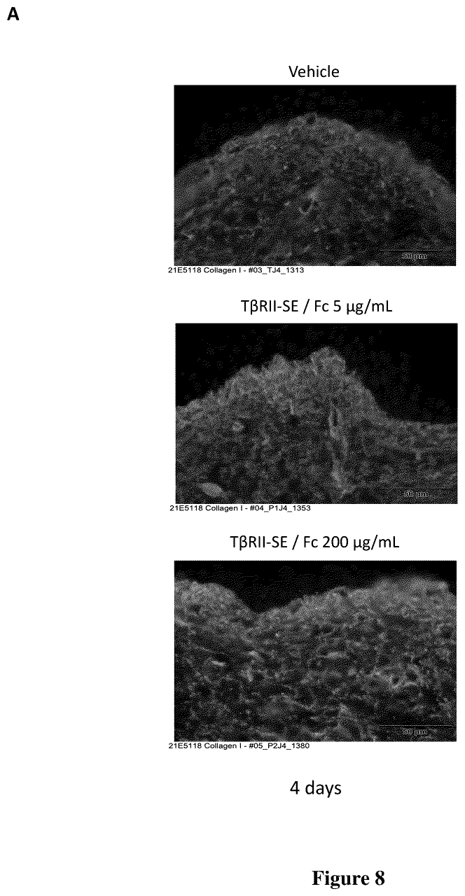

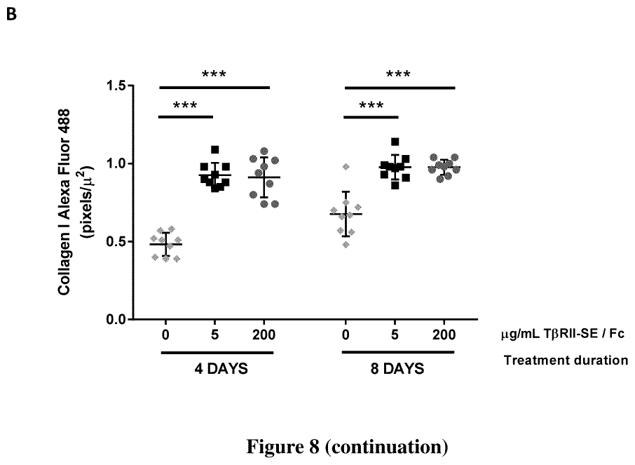

The TβRII-SE/Fc fusion protein also exhibited statistically significant anti-aging activity (p<0.001) when stimulating collagen I production after 4 and 8 days of treatment (FIG. 8).

TβRII-SE/Fc induced a significant increase in the morphology, migration, proliferation, cell adhesion and differentiation of the neoepidermis in damaged skin explants.

TβRII-SE/Fc also induced a significant increase in collagen I production in the dermis.

Example 1: TβRII-SE/Fc production and use of TβRII-SE/Fc to treat cutaneous and pulmonary fibrosis TβRII-SE/Fc was produced according to the methods described in Marcela Bertolio, et., al., Frontiers in Cell and Developmental Biology. 9:690397.doi: 10.3389/fcell.2021.690397 (2021).

TβRII-SE/Fc (5.2 mg/ml) was diluted in PBS pH 7.4 according to the study protocol. Nintedanib which was purchased from Chemexpress Co., Ltd. (China). To prepare the dosing solution, Nintedanib was weighed and resuspended in 1% methylcellulose.

Route and dose of administration of the composition: TβRII-SE/Fc was administered intravenously in a volume of 5 ml/kg (5 mg/kg) twice a week (day 7, 10, 14 and 17) from day 7 to 20 (4 doses) in the pulmonary fibrosis model and until day 28 (6 doses) in the cutaneous fibrosis model. Nintedanib was administered orally in a volume of 10 ml/kg (100 mg/kg) once daily from day 7 to 20 in the pulmonary fibrosis model and until day 28 in the skin fibrosis model.

Animals: Thirty 6 weeks old female C57BL/6J mice were obtained from Japan SLC, Inc. (Japan). These mice were housed and fed a normal diet (CE-2; CLEA Japan) under controlled conditions. All the animals used in this study were cared for following the following guidelines: 1) Animal Welfare and Handling Law (Ministry of the Environment of Japan, Law No. 105 of Oct. 1, 1973); 2) Standards Relating to the Care and Handling of Laboratory Animals and Pain Relief (Notice No. 88 of the Ministry of the Environment of Japan, Apr. 28, 2006); 3) Guidelines for the proper conduct of animal experiments (Scientific Council of Japan, June 1, 2006).

The animals were kept in an SPF facility under controlled conditions of temperature (23±3° C.), humidity (50 ±20%), lighting (12-hour artificial light and dark cycles; light from 8:00 to 20:00) and air exchange. A high pressure was maintained in the experimental room to avoid contamination of the facility.

Animals were housed in a TPX cage (CLEA Japan) with a maximum of 5 mice per cage. Clean sterilized paper (Japan SLC) was used for the bedding of the animals and replaced once a week. Mice were identified by ear puncture. Each cage was also given a specific identification code.

The normal sterilized diet was provided ad libitum, placed in a metal lid at the top of the cage. RO water was also supplied ad libitum from a water bottle fitted with a rubber stopper and a sipper tube. The water bottles were replaced once a week, cleaned, autoclaved, and reused.

Induction of the BLM-induced lung fibrosis mouse model: On day 0, the development of lung fibrosis was induced in 30 mice by a single intratracheal administration of bleomycin hydrochloride (BLM, Nippon Kayaku, Japan) in saline at a dose 3.0 mg/kg, in a volume of 50 pL per animal using Microsprayer (Penn-Century, USA).

Induction of BLM-induced skin fibrosis model: Mice were randomized into 3 groups of 10 mice based on their body weight the day before BLM administration. On day 0, 30 mice were administered subcutaneously BLM (lot #391870, Nippon Kayaku, Japan) in saline at a dose of 50 μg/mouse, in a volume of 50 μl every other day (day 0, 2 , 4, 6, 8, 10, 12, 14, 16, 18, 20, 22, 24 and 26).

Animal monitoring and sacrifice: Viability, clinical signs and behavior were monitored daily. Body weight was recorded daily from day 0. Dosing volume was adjusted based on last body weight. Mice were observed for significant clinical signs of toxicity, morbidity, and mortality approximately 60 minutes after each administration. Animals were sacrificed on day 21 (pulmonary fibrosis model) and day 28 (model of cutaneous fibrosis by exsanguination through the abdominal vena cava under a mixture of medetomidine (0.75 mg/kg), midazolam (4 mg/kg) and butorphanol (5 mg/kg) anesthesia. The timing of dosing and termination was recorded. The weight of the left and post-cava lobes of all mice was recorded.

Collection of samples: The bronchi of the left lobe and post cava were ligated to avoid the leak of the fixative. The permanent needle was inserted into the trachea and connected to the instillation path of the syringe. The syringe was loaded with 10% neutral buffered formalin and held at a height of 20 cm. The upper (a), middle (b) and lower (c) lobes were then instilled with 10% neutral buffered formalin and ligated after inflation. Three fixed lobes were harvested, the unfixed left lung (e) and the unfixed pos cava lobe (d). Two unfixed lobes were washed with cold saline and weight was measured.

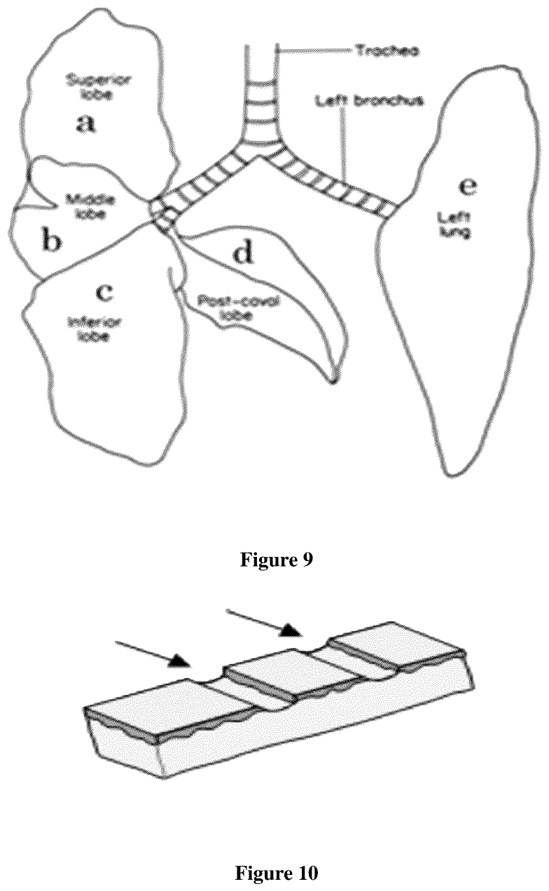

Three lobes were fixed in 10% neutral buffered formalin for 24 hours. After fixation, these samples were embedded in paraffin for Masson's trichrome staining. The postcaval lobe (D) was flash frozen in liquid nitrogen and stored at −80° C. for further analysis. The left lung (E) was instantly frozen in liquid nitrogen and stored at −80° C. for further analysis (see FIG. 9)

For skin samples, a previously shaved back sample was collected and fixed in 10% neutral buffered formalin for 24 hours. After fixation, these samples were embedded in paraffin for histopathological analysis.

Histopathological analysis: Sections of paraffin blocks from skin and right lung tissue pre-fixed in 10% neutral buffered formalin were sectioned at 4 μm using a rotary microtome (Leica Microsystems).

For H&E staining, sections of pre-fixed skin tissue paraffin blocks were cut in 10% neutral buffered formalin and stained with hematoxylin of Lillie-Mayer (Muto Pure Chemicals Co., Ltd., Japan) and eosin solution (FUJIFILM Wako Pure Chemical Corporation, Japan). For quantitative analysis of dermal thickness, bright-field images of the HE-stained section were captured with a digital camera (DFC295; Leica, Germany) at 100-fold magnification, and dermal thickness was measured in 5 fields/section using ImageJ software (National Institute of Health, USA).

For Masson's trichrome staining of both skin and lung, sections were deparaffinized and rehydrated, followed by re-fixation with Bouin's solution for 15 minutes. Sections were stained in Weigert's iron hematoxylin working solution (Sigma-Aldrich), scarlet-fuchsia Biebrich's solution (Sigma-Aldrich), phosphotungstic acid/phosphomolybdic acid solution, aniline blue solution, and acetic acid solution al 1% (Sigma-Aldrich). For quantitative analysis of the area of pulmonary fibrosis, bright-field images of Masson's trichrome stained sections were randomly captured using a digital camera (DFC295) at 100-fold magnification, and the subpleural regions in 20 fields/mouse were captured. evaluated according to the classification criteria for pulmonary fibrosis (Aschcroft T. et al., J Clin Pathol, 1988; 41: 467-70). All sections were blindly analyzed by an experimenter.

Grades of pulmonary fibrosis (Aschcroft score):
Normal lung or minimal fibrous thickening of the alveolar or bronchiolar walls.
2-3 Moderate thickening of the walls without obvious damage to the lung architecture.
4-5 Increased fibrosis with definite damage to the lung structure and formation of fibrous bands or small fibrous masses.
6-7 Severe distortion of structure and large fibrous areas; lung in “honeycomb”
8 Total fibrous obliteration of the field.

For the Ashcroft score, the raw data was calculated as an integer. The average value of 20 fields in each section (lobes, upper, middle and bottom) was taken as the Ashcroft score of each individual animal and calculated to the first decimal place. The mean value and standard deviation of each group were rounded to the second decimal place and calculated to the first decimal place.

Statistical analysis was performed using the ANOVA method or the Bonferroni multiple comparison test on GraphPad Prism 7 (GraphPad Software Inc., USA). The model group was used as a control group for multiple comparisons (Dunnett's test). Values of p<0.05 were considered statistically significant. A trend was assumed when P values<0.1. The results were expressed as mean ±SD.

### Design and Treatment Scheme

### Skin Fibrosis Model

### Group 1: Vehicle

Ten mice with BLM-induced skin fibrosis were administered intravenously phosphate buffered saline (PBS) pH 7.4 vehicle in a volume of 5 ml/kg twice a week (Monday and Friday) from day 0 to 27 (8 doses in total).

### Group 2: TβRII-SE/Fc

Ten mice with BLM-induced skin fibrosis were administered intravenously vehicle supplemented with TβRII-SE/Fc at a dose of 5.0 mg/kg in a volume of 5 ml/kg twice a week (Monday and Friday) from day 0 to 27 (8 doses in total).

### Group 3: Nintedanib

Ten mice with BLM-induced skin fibrosis were orally administered 1% methylcellulose supplemented with Nintedanib at a dose of 100 mg/kg in a volume of 10 ml/kg once daily from day 0 to 27.

Pulmonary fibrosis model: After one week of administration of bleomycin (BLM), the animals were randomized into three experimental groups according to changes in body weight:

### Group 1: Vehicle

Ten mice with BLM-induced pulmonary fibrosis were administered intravenously phosphate buffered saline (PBS) vehicle pH 7.4 in a volume of 5 ml/kg twice weekly from day 0 to 20 (4 doses).

### Group 2: TβRII-SE/Fc

Ten mice with BLM-induced pulmonary fibrosis were administered intravenously the vehicle supplemented with TβRII-SE/Fc at a dose of 5.0 mg/kg in a volume of 5 ml/kg twice a week from day 7 to 20 (4 doses).

### Group 3: Nintedanib

Ten mice with BLM-induced pulmonary fibrosis were orally administered 1% methylcellulose supplemented with Nintedanib at a dose of 100 mg/kg in a volume of 10 ml/kg once daily from day 7 to 20.

### Example 2: Use of TβRII-SE/Fc for the Treatment of Liver Fibrosis

TβRII-SE/Fc was tested at 1 and 5 mg/kg. The 5.2 mg/ml stock solution stored at −80° C. was thawed on ice and kept at 4° C. Before dosing, the stock solution was diluted in 1 mg/ml or 0.2 mg/ml working solution with sterile PBS.

Route and dose of administration of the composition: TβRII-SE/Fc was administered intravenously in a volume of 5 ml/kg at 1 and 5 mg/kg twice a week from week 3 to 6 (8 doses).

Animals: 42 male Sprague Dawley rats, SPF grade, from the Shanghai Laboratory Animal Center (SLAC) of the Chinese Academy of Sciences, from 180-200 grams of body weight. Normal food was provided ad libitum throughout the study and the animals had free access to drinking water.

After arrival, the animals were housed for acclimatization in specific pathogen free (SPF) facilities. The housing conditions were temperature of 20-26° C., humidity of 40-70% and a cycle of 12 hours of light/12 hours of darkness. After 7 days, blood was collected and serum was prepared by centrifugation at 5,000 rpm for 10 minutes and then used for ALT/AST measurement.

Induction of hepatic fibrosis by CCl4 in rats: Hepatic fibrosis was induced by carbon tetrachloride (CCl4) 25% in olive oil, administered by oral gavage three times a week (TIW). After acclimatization, 32 rats received CCl4 at a dose of 0.5 ml/kg (795 mg/kg), in a volume of 1 ml, by oral gavage, 3 times a week (TIW) for 2 weeks. Instead, 10 rats received vehicle (olive oil) (control group). After two weeks of treatment, 2 control animals were sacrificed and fibrosis was confirmed by Sirius red staining.

Animal monitoring and sacrifice: Viability, clinical signs and behavior were monitored daily. Body weight was recorded weekly. The animals were sacrificed after 6 weeks of study.

Specimen Collection: Blood was collected at weeks 0 and 2 for ALT/AST measurement. Serum samples were prepared by centrifugation at 5,000 rpm for 10 min.

Upon completion, blood was drawn by cardiac puncture and serum was prepared as before. The livers were dissected and weighed. After photographing, the left lobe was fixed in formalin and then transferred to the histopathology laboratory for H&E staining.

ALT/AST measurement: Serum ALT and AST were measured with an automated biochemical analyzer (MINDRAY BS-380).

Histopathological analysis: Sections of paraffin blocks from liver tissue pre-fixed in 10% neutral buffered formalin and sectioned at 4 μm using a rotary microtome (Leica Microsystems).

For H&E staining, sections were deparaffinized and rehydrated, then stained with hematoxylin solution, placed in 0.25% HCl-alcohol, counterstained in eosin, and dehydrated and mounted on a coverslip. An expert blindly analyzed sections of the slides for them; degrees of fibrosis, inflammatory cell infiltrates, steatosis, degeneration/necrosis of hepatocytes.

The scoring was done as follows:

Degrees of fibrosis:
0—No fibrosis.
1. Fibrous expansion with short fibrous septa.
2. Fibrous expansion of most centrilobular areas, with or without short fibrous septa.
3. Fibrous expansion with occasional bridging fibrosis between centrilobular and/or portal areas.
4. Fibrous expansion with bridging fibrosis between centrilobular and/or portal areas.
5. Bridging fibrosis between centrilobular and/or portal areas, with occasional nodules (incomplete cirrhosis).
6. Cirrhosis (probable or definitive), severe bridging fibrosis with nodules.

General histopathology classification scheme:

0—No notable findings; 1—Minimum; 2—Light; 3—Moderate; 4—Marking; 5—Severe;

Statistical analysis was performed using ANOVA (parametric) or Kruskal-Wallis (non-parametric) tests as appropriate, using Graph Pad Prism 7 software. (Graph Pad Software Inc., USA). The model group was used as a control group for multiple comparisons (Dunnett's or Dunn's tests). Values of p<0.05 were considered statistically significant. A trend was assumed when P values <0.1. The results were expressed as mean ±SD.

Design and treatment scheme: After 2 weeks of CCl4 treatment, the 30 rats were randomly assigned to 3 groups based on their serum ALT/AST levels. CCl4 or vehicle was administered continuously until week 6. TβRII-SE/Fc was administered 2 hours before treatment with CCl4 twice weekly (BIW) with 5 ml/kg by intravenous injection as follows:
Group 1 (Vehicle): Vehicle (PBS pH 7.4) was administered intravenously to ten healthy animals in a volume of 5 ml/kg BIW from week 3 to 6 (8 doses). The olive oil by oral gavage TIW was administered continuously until the end.
Group 2 (CCl4 model): Ten rats with CCl4-induced liver fibrosis received vehicle intravenously (PBS pH 7.4) in a volume of 5 ml/kg BIW from week 3 to 6 (8 doses). 795 mg/kg CCl4 was continuously administered by TIW oral gavage until completion.
Group 3 (high dose): Ten rats with CCl4-induced liver fibrosis were administered intravenously TβRII-SE/Fc at a dose of 5 mg/kg in a volume of 5 ml/kg BIW from week 3 to week 6 (8 doses). 795 mg/kg CCl4 was continuously administered by TIW oral gavage until completion.
Group 4 (low dose): ten rats with CCl4-induced liver fibrosis were administered intravenously TβRII-SE/Fc at a dose of 1 mg/kg in a volume of 5 ml/kg BIW from week 3 to week 6 (8 doses). 795 mg/kg CCl4 was continuously administered by TIW oral gavage until completion.

### Example 3: Use of TβRII-SE/Fc for the Treatment and Repair of Wounds; and to Decrease Aging in Living Human Skin Explants

The aim of the study was to evaluate the effects of two concentrations of TβRII-SE-/Fc on wound healing and skin aging using live human skin explants.

The wound healing effect was evaluated by: analysis of the general morphology of the wounded area and the non-wounded area with a focus on the growth bud,

An analysis of the length of the growth bud.

The anti-aging effect has been evaluated by: collagen I immunostaining

TβRII-SE/Fc was assayed at 5 (P1) and 200 (P2) μg/kg. The 5.2 mg/ml stock solution stored at −80° C. was thawed on ice and kept at 4° C. Before dosing, the stock solution was diluted in culture medium. All dilutions were prepared extemporaneously each time the treatment was renewed.

Explant preparation: In a tummy tuck from a 55-year-old Caucasian woman (reference: P2422-AB55), 21 circular skin explants (12 mm in diameter) and 21 rectangular skin explants (10×15 mm) were prepared. The explants were kept in survival in BEM culture medium (BIO-EC explant medium) at 37° C. in a humid atmosphere of 5% CO2.

Distribution of explants: The explants were distributed in 8 lots as follows: Table 1.

Performance of mechanical lesions: In each rectangular explant, for batches B0, B, BP1 and BP2, two mechanical lesions were performed on day 0 with a 3.5 mm diameter biopsy punch (see FIG. 10).

| | | | Number of | Sampling |
|---|---|---|---|---|
| Batch | Designation | Treatment | explants | time |
| T0 | Tissue control | — | 3 | Day 0 |
| B0 | Wound control | Mechanical injury | 3 | Day 0 |
| T | Control | — | 6 | Day 4, |
| | | | | Day 8 |
| P1 | Product 1 | A at 5 μg/mL | 6 | Day 4, |
| | | | | Day 8 |
| P2 | Product 2 | A at 200 μg/mL | 6 | Day 4, |
| | | | | Day 8 |
| B | Wound | Mechanical injury | 6 | Day 4, |
| | | | | Day 8 |
| BP1 | Wound + Product 1 | Wound + A at | 6 | Day 4, |
| | | 5 μg/mL | | Day 8 |
| BP2 | Wound + Product 2 | Wound + A at | 6 | Day 4, |
| | | 200 μg/mL | | Day 8 |

Treatment: Products P1 and P2 were incorporated into the culture medium of the batch in question on day 0 (D 0), D 2, D 4 and D 6. Half of the culture medium (1 ml) was renewed to D 2, D 4 and D 6. Control explants received no treatment.

Sampling and measurement of anti-aging activity: On day D0, the 3 explants from the T0 lots were collected and cut into two parts. Half was fixed in buffered formalin and the other half was frozen at −80° C. In D4 and D8, 3 explants from lots T, P1 and P2 were collected and processed in the same way.

Assessment of wound repair activity: On day D0, the 3 explants from batches B0 were harvested and cut into three parts. The first part was fixed in buffered formalin, the second part was frozen at −80° C. and the third part was stored as RNA for eventual later transcriptomic analysis. In D4 and D8, 3 explants from lots B, BP1 and BP2 were collected and processed in the same way.

Histological studies: After fixation for 24 hours in buffered formalin, the samples were dehydrated and embedded in paraffin using a Leica TP 1010 automatic dehydration equipment. The samples were then included using a Leica embedding station EG 1160. 5 μm thick sections were made using a Leica RM 2125 Minot type microtome and then mounted on Superfrost® histological glass slides. Microscopic observations were made using a Leica DMLB or Olympus BX43 microscope. The images were digitized with a digital Olympus DP72 camera with CellD storage software.

General morphology: The observation of the general morphology of the epidermal and dermal structures, as well as the growth bud, was carried out after staining of paraffin sections according to Masson's trichrome, Goldner variant.

Collagen I immunostaining: Collagen I immunostaining was performed on frozen skin sections with a polyclonal anti-collagen I antibody (Monosan, ref. PS047), diluted 1:50 in PBS-0.3% BSA and incubated for 1 hour at room temperature. Staining was revealed by AlexaFluor 488 (Life Technologies, ref. A11008). The nuclei were subsequently stained with prodidium iodide. Staining was done manually and evaluated by microscopic observation.

1. A method of treating fibrosis in a mammal in need thereof, wherein the method comprises administering to said mammal a therapeutically effective amount of a fusion protein comprising a soluble TGF-β type II receptor bound to a stabilizing agent.

2. The method according to claim 1, wherein the soluble TGF-β type II receptor has at least 85% sequence identity with the amino acid sequence shown in SEQ ID No. 1.

3. The method according to claim 1, wherein the stabilizing agent comprises the Fc domain of a human immunoglobulin.

4. The method according to claim 1, wherein the fibrosis is selected from the group consisting of liver fibrosis, lung fibrosis and skin fibrosis.

5. The method according to claim 1, wherein the mammal is a human.

6. The method according to claim 5, wherein the human has a selected disorder or disease of non-alcoholic steatohepatitis (NASH), Scleroderma/Systemic Sclerosis (SSc), idiopathic pulmonary fibrosis, and Primary biliary cholangitis (PBC).

7. The method according to claim 1, wherein the amino acid sequence of the fusion protein has at least 85% identity with the sequence shown in SEQ ID No. 2.

8. A composition for the treatment of fibrosis, wherein said composition comprises a soluble type II TGF-β receptor linked to a stabilizing agent, and pharmaceutically acceptable excipients; wherein fibrosis is selected from the group consisting of liver fibrosis, lung fibrosis, and skin fibrosis.

9. A method for healing wounds in a mammal in need thereof, wherein the method comprises administering to said mammal a therapeutically effective amount of a fusion protein comprising a soluble TGF-β type II receptor bound to a stabilizing agent.

10. The method according to claim 9, wherein the soluble TGF-β type II receptor has at least 85% sequence identity with the amino acid sequence shown in SEQ ID No. 1.

11. The method according to claim 9, wherein the stabilizing agent comprises the Fc domain of a human immunoglobulin.

12. The method according to claim 9, wherein the amino acid sequence of the fusion protein has at least 85% identity with the sequence shown in SEQ ID No. 2.

13. The method according to claim 9, wherein the mammal is a human.

14. A composition for wound healing, wherein said composition comprises comprising a soluble TGF-β type II receptor bound to a stabilizing agent; and pharmaceutically acceptable excipients.

15. A method for slowing the aging of the skin of a human, wherein the method comprises administering to said human an effective amount of a fusion protein comprising a soluble TGF-β type II receptor bound to a stabilizing agent.

16. The method according to claim 15, wherein the soluble TGF-β type II receptor amino acid sequence has at least 85% sequence identity with the amino acid sequence shown in SEQ ID No. 1.

17. The method according to claim 15, wherein the stabilizing agent comprises the Fc domain of an immunoglobulin.

18. The method according to claim 15, wherein the amino acid sequence of the fusion protein has at least 85% identity with the sequence shown in SEQ ID No. 2.

19. A cosmetic composition for reducing the aging of the skin of a human being, wherein said composition comprises comprising a soluble TGF-β type II receptor linked to a stabilizing agent; and acceptable excipients.

20-22. (canceled)
